(19) United States
(12) Patent Application Publication
(10) Pub. No.: US 2016/0310753 A1
(43) Pub. Date: Oct. 27, 2016

(54) BODY TUNER SYSTEM

(71) Applicant: Brian Bravo, Porter Ranch, CA (US)

(72) Inventor: Brian Bravo, Porter Ranch, CA (US)

(21) Appl. No.: 15/133,152

(22) Filed: Apr. 19, 2016

Related U.S. Application Data
(63) Continuation-in-part of application No. 14/311,254, filed on Jun. 21, 2014, now abandoned.
(60) Provisional application No. 61/837,819, filed on Jun. 21, 2013.

(51) Int. Cl. A61N 1/40; H02J 50/00

(52) U.S. Cl. CPC A61N 1/40; H02J 50/00

(57) ABSTRACT

A body tuning device having at least one power source, a switch to control operation of the body tuning device, at least one electrical transformer, at least one spark gap with at least two separated tungsten diodes, a plurality of resistors and capacitors, conductive material extending between each component and coiled around a first and a second concentric tube, and one or more probes extending from each end of the second tube. The at least one transformer is in electrical communication between the at least one spark gap and the at least one power source. The at least one spark gap is in electrical communication with the one or more resistors. The capacitors discharge the flow of electricity through the one or more probes. The flow of electricity through the one or more probes is configured to adjust a cellular electrical environment of a user.

### CROSS-REFERENCE TO RELATED APPLICATION(S)

The present application claims priority to U.S. Non-Provisional patent application Ser. No. 14/311,254 filed on Jun. 21, 2014, entitled “HIGH-FREQUENCY CLIMBING SCALAR ARC/BODY TUNER SYSTEMS”, which claims priority from prior provisional application Ser. No. 61/837,819, filed Jun. 21, 2013, the entire disclosure of which is incorporated by reference herein.

### BACKGROUND OF THE INVENTION

1. Field of Invention

The present invention relates to the field of medical devices, more particularly to body tuning devices.

2. Description of Related Art

On a cellular level, organisms operate one require electrical impulses and potentials. Action potential and electrochemical gradients are essential for the proper functionality of a cell and therefore of the organism as a whole. When the electrical balance is out of standard levels the cell ceases to operate at optimum functions.

The human body is susceptible to a myriad of different external stimuli having varying effects. Often, these effects have a negative impact on an individual's health. For example, people are exposed to sources of radiation daily. Some examples of these sources include computers, cell phones, and hair dryers. Overexposure to radiation can cause fatigue, lack of energy, weakened immune systems, and changes in physical appearance.

Conventional treatment results in a painful shock, or are ineffective completely. A lack of control and effective induction or conversion of electrical energy has resulted in a lack of appreciation for the beneficial effects of electrotherapies.

Based on the foregoing, there is a need in the art for an effective device to treat and manage the aforementioned negative effects of external stimuli. A device that is safe for human use and provides efficient relief for the various ailments caused by an altered cellular electrical environment.

### SUMMARY OF THE INVENTION

A body tuning device having at least one power source, a switch to control operation of the body tuning device, at least one electrical transformer, at least one spark gap with at least two separated tungsten diodes, a plurality of resistors and capacitors, conductive material extending between each component and coiled around a first and a second concentric tube, and one or more probes extending from each end of the second tube. The switch is in electrical communication with the body tuning device. The at least one transformer is in electrical communication between the at least one spark gap and the at least one power source. Voltage is increased by the at least one electrical transformer. The at least one spark gap is in electrical communication with the one or more resistors. Electricity from the power source flows through the body tuner to the capacitor. The capacitors discharge the flow of electricity through the one or more probes. The flow of electricity through the one or more probes is altered and configured to adjust a cellular electrical environment of a user.

In an embodiment, the body tuner has one or more attachments in communication with the one or more probes; the one or more attachments are configured to contour a user.

In an embodiment, one or more of the attachments are selected from the group consisting of a frusticonical rod, a toroidial wand, and a plate surface.

In an embodiment, the switch is a control module having at least one processor and a plurality of buttons. The control module is in communication with the body tuner, wherein the control module is configured to adjust the electrical attributes of the flow of electricity.

In an embodiment, the flow of electricity between the one or more probes creates an arc, and wherein an electromagnetic environment affects the arc.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a view of the circuit diagram showing the various components of the body tuner system, according to an embodiment of the present invention;

FIG. 2 is a detailed perspective view of the body tuner system, according to an embodiment of the present invention.

FIG. 3A is a perspective view of the body tuner system, according to an embodiment of the present invention.

FIG. 3B is a perspective view of the body tuner system, according to an embodiment of the present invention.

FIG. 3C is a perspective view of the body tuner system, according to an embodiment of the present invention.

### DETAILED DESCRIPTION OF PREFERRED EMBODIMENTS

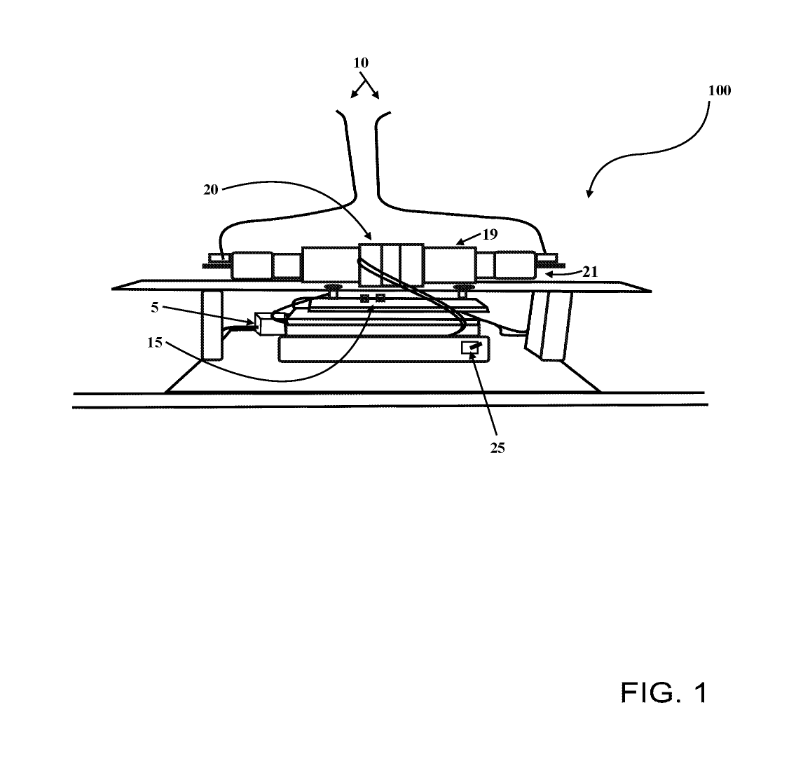

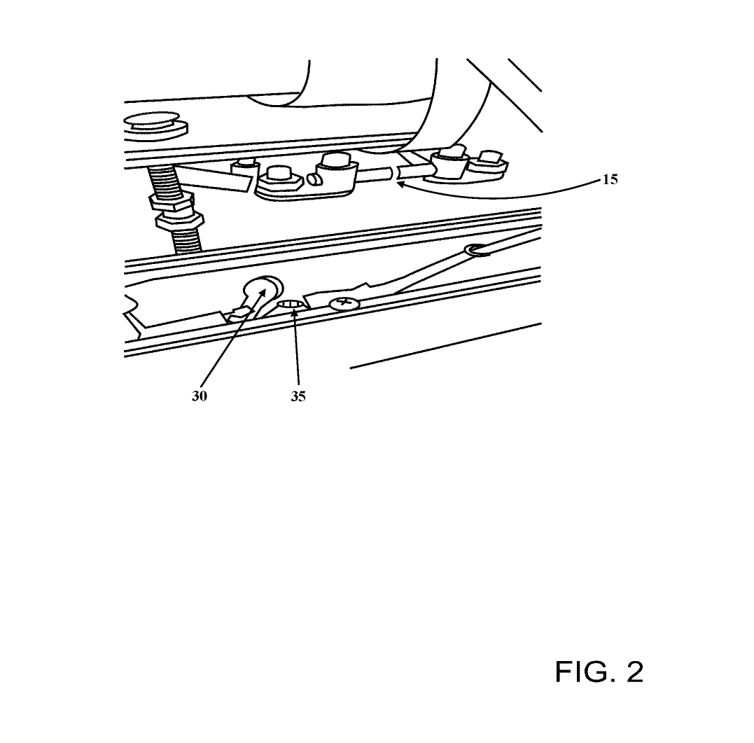

Preferred embodiments of the present invention and their advantages may be understood by referring to FIGS. 1-3, wherein like reference numerals refer to like elements.

A body tuning system has a power source electrically connected to a voltage transformer 5. The voltage transformer 5 has a plurality of ports adapted to allow for ingress and egress of electrical connections such as wires. A switch 25 is in circuit with the power source and the transformer 5 to manage the operation of the body tuner system 100. At least one of the transformer ports has at least one wire extending therefrom. One end of the wire is connected to the transformer 5 and the other end of the wire is electrically connected to at least one side of a spark gap 15.

In an embodiment, the spark gap 15 is made of two or more conductive nodes. Conductive material such as tungsten is use for each node and the spacing of the gap 15 is sufficient to allow for visualization of gaseous ionization and a visual current passing between the nodes.

In an embodiment, the body tuner system 100 has a one or more tubes 20, wound with a 24-gauge magnet wire. Also integrated into the device is at least one high voltage (HV) transformer 5, at least one HV capacitor 30, one or more bleeder resistors 35, two or more electrodes/probes 10, at least one tungsten spark gap 15, and at least one toggle switch 25. The capacitor 30 can be enclosed in a non conductive material for precautionary measures. When in use, the device can magnify and convert electromagnetic energy potentials from the air and create scalar energy. The scalar energy can then be released from the two electrodes 10.

In an embodiment, the body tuning system is made of materials such as wood, and other non-conductive material, transparent thermoplastic, and other conductive material, or other suitable materials known to one skilled in the art.

Referring to the drawings by numerals of reference there is shown in FIGS. 1-3, various views of high-frequency climbing scalar arc/body tuner system 100 comprising a tube 20 wound with a 24-gauge magnet wire; an HV transformer 5; an HV capacitor 30, bleeder resistors 35; two electrodes 10; a tungsten spark gap 15; and a toggle switch 25; wherein the high-frequency climbing scalar arc/body tuner system 100 comprises in functional combination the tube 20 wound with the 24-gauge magnet wire, the HV transformer 5, the HV capacitor 30, the bleeder resistors 35, the two electrodes 10, the tungsten spark gap 15; and the toggle switch 25.

In an embodiment, the body tuner system 100 is powered via 110V and steps up to at least 100,000 volts. High-frequency climbing scalar arc/body tuner system 100 causes voltage to be increased and current decreased thereby minimizing electrocution hazard. As such, high-frequency climbing scalar arc/body tuner system 100 provides that since the current is lowered, the current is thereby able to crawl over skin rather than penetrating through the skin of a user to provide at least one medically useful result.

In an embodiment, the HV capacitor 30 may be enclosed in wood for precautionary measures related to safety for the user. Other suitable non-conductive materials may be used. The body tuner system 100 is configured to pull in electromagnetic energy from air and convert this energy to medically acceptable energy in the form of scalar arcs to interact with a user such that the energy transformed by the body tuner system 100 corrects an imbalance of the user's cellular electrical environment. The scalar energy is able to be released from the two electrodes/probes 10. More electrodes/probes 10 may be used in alternative embodiments.

In an alternative embodiment, the body tuner system is able to provide power to nearby appliances through the electromagnetic field created by the system. For example, a light bulb placed in close proximity to the electrodes/probes 10 will be energized from the electrical filed produced through the electricity flow through the electrodes/probes 10.

In an alternative embodiment, the high-frequency climbing scalar arc/body tuner system 100 is adapted to be used to repair damaged human cells by increasing the human cells to a healthy electrical environment, for example 70-90 millivolts. In another embodiment use high-frequency climbing scalar arc/body tuner system 100 is adapted to track entities. Further, high-frequency climbing scalar arc/body tuner system 100 is useful to help increase health of bloods cells of said user by boosting energy levels. Those with ordinary skill in the art will now appreciate that upon reading this specification and by their understanding the art of electricity use as described herein, methods of high-frequency electricity use will be understood by those knowledgeable in such art.

In an embodiment, the body tuner system 100 is part of a kit having at least body tuner 100, and at least one set of user instructions, and one or more attachments 41, 42, 43 configured for user application. The kit has instructions such that functional relationships are detailed in relation to the structure of the invention (such that the invention can be used, maintained, or the like in a preferred manner). High-frequency climbing scalar arc/body tuner system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different powering combinations, parts may be sold separately, etc., may be sufficient.

In use, the power source energizes the body tuner system 100. Electricity is routed through a system of conductive material between the power source and downstream components. The transformer 5 converts the voltage to an increased value for supply of a medically effective amount. The electricity is then routed through the spark gap 15. The spark gap 15 indicates ionization of gas existing between the two nodes of the gap 15. For example, when the voltage difference between the conductors exceeds the breakdown voltage of the gas within the gap, a spark forms, ionizing the gas and drastically reducing its electrical resistance. An electric current then flows until the path of ionized gas is broken or the current reduces below a minimum value called the holding current. This usually happens when the voltage drops, but in some cases occurs when the heated gas rises, stretching out and then breaking the filament of ionized gas. The visual ionization of the gas, producing a spark also acts to indicate an integral step in the initiation of the body tuner system 100. After the electricity has extended across the spark gap 15, it continues to at least one resistor 35. The at least one resistor 35 provides a known resistance for the desired control of the electrical attributes such that the current and voltage leaving the resistor 35 are acceptable. This electricity, after the resistor 35, enters at least one capacitor 30 or a known farad value. The sorted energy within the plurality of capacitor 30 plates continues to be stored until a set threshold. In an embodiment, the set threshold is adjustable and managed by the user. When the threshold is reached, the capacitors 30 are discharged across a plurality of coils or conductive material. There are at least two sets of conductive material coiled around a non-conductive and generally annular tube 20. A first coil about a tube 19 has a diameter larger than a second coil. A second coil has a smaller diameter and the second tube 21 having the plurality of coils extends through the first coil. This configuration allows for the creation of a magnetic field with appropriate direction of electrical current. The coils comprise the same continuous length of conductive material. One or more probes 10 extend, on a same plane, from each end of the second tube 21. A user is then extends an appendage through the space between each of the probes 10. Current flowing through the probes 10 to a terminal end provides for an electro-environment penetrating and altering the magnetic and electrical potential within the users cellular structure.

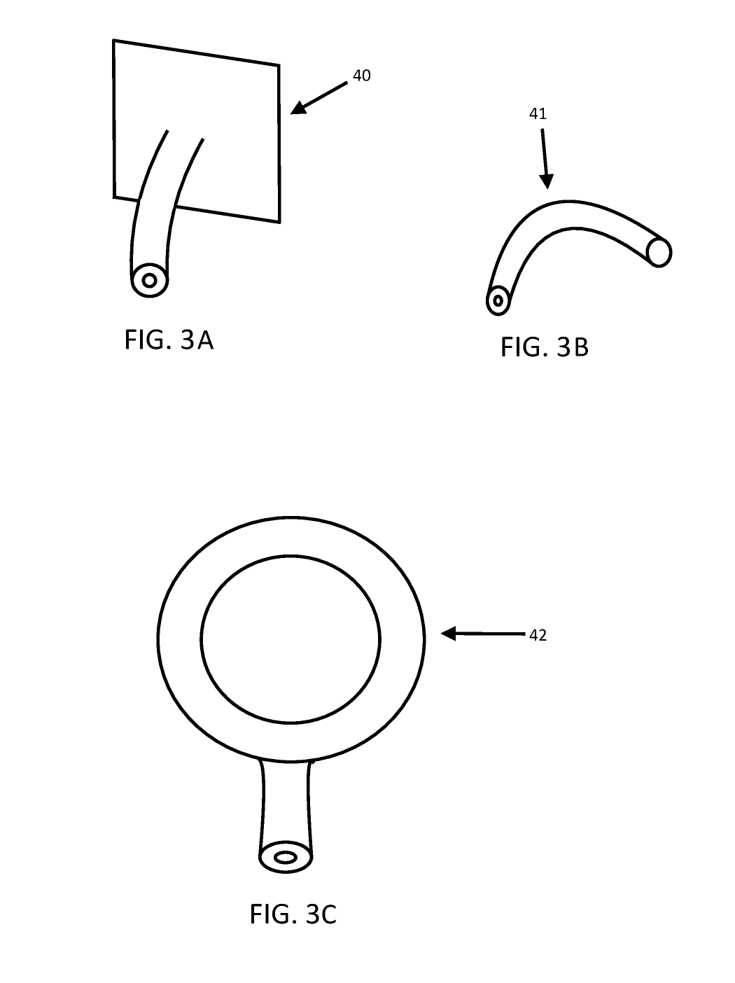

In an alternative embodiment, illustrated in FIGS. 3A, 3B, and 3C, the one or more probes 10 are configured to attach to one or more attachments 40, 41, 42. Examples of select attachments would include extensions of the probes 10 in various shapes such as various parabolic curves 41, flat plates 40 with large surface area, and rings 42. The s may include ceramic components where appropriate as a non-conductive buffer for user contact.

In an alternative embodiment, a control module having at least one power source and at least one processor is in communication with the body tuner system 100. The control module has a plurality of button corresponding to on or more values for user control, wherein a user is able to select, adjust, or allow for automated control of capacitor 30 discharge and voltage regulation depending of specific medical use.

In an alternative embodiment, the body tuner system 100 is reactive to various environmental electromagnetic phenomena. External forces affect the current and any visible arching between the one or more probes 10. For example, when a deviation in levels of electromagnetic environment exists, the electrical arc is altered. The amount of deviation or alteration from baseline operation corresponds to the level and value of the environmental electromagnetic phenomena.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A body tuning device comprising:
a. at least one power source;
b. a switch controls operation of the body tuning device, wherein the at least one switch is in electrical communication with the body tuning device;
c. at least one electrical transformer;
d. at least one spark gap having at least two separated tungsten diodes;
e. one or more resistors;
f. one or more capacitors;
g. conductive cord in electrical communication with the one or more capacitors, wherein the conductive cord is coiled around a first tube and a second tube, wherein the first tube and second tube are concentric;
h. one or more probes extending from each end of the second tube, wherein the at least one transformer are in electrical communication between the at least one spark gap and the at least one power source, wherein voltage is increased by the eat least one electrical transformer, wherein the at least one spark gap is in electrical communication with the one or more resistors, wherein electricity from the power source flows through the body tuning device to the capacitor, wherein the capacitors discharge the flow of electricity through the one or more probes, wherein the flow of electricity through the one or more probes is altered, and wherein the altered flow of electricity is configured to adjust a cellular electrical environment of a user.

2. The body tuning device of claim 1, further comprising one or more attachments in communication with the one or more probes, wherein the one or more attachments are configured to contour a user.

3. The body tuning device of claim 2, wherein the one or more attachments are selected from the group consisting of a frusticonical rod, a toroidial wand, and a plate surface.

4. The body tuning device of claim 1, wherein the switch is a control module having at least one processor and a plurality of buttons, wherein the control module is in communication with the body tuning device, wherein the control module is configured to adjust the electrical attributes of the flow of electricity.

5. The body tuning device of claim 1, wherein the flow of electricity between the one or more probes creates an arc, and wherein the arc is affected by an electromagnetic environment.
